(19) United States
(12) Patent Application Publication
(10) Pub. No.: US 2004/0106678 A1
(43) Pub. Date: Jun. 3, 2004

(54) Compositions for the parenteral administration of calcium and magnesium

(76) Inventors: Thomas A. Dobbins, Howard, OH (US); David B. Wiley, Warsaw, OH (US); Michael Davis, Glen Rose, TX (US)

Correspondence Address: THOMPSON HINE L.L.P., 2000 COURTHOUSE PLAZA , N.E., 10 WEST SECOND STREET, DAYTON, OH, 45402, US

(21) Appl. No.: 10/667,283

(22) Filed: Sep. 17, 2003

Related U.S. Application Data
(60) Provisional application No. 60/411,229, filed on Sep. 17, 2002.

(51) Int. Cl. A61K 31/19

(52) U.S. Cl. 514/557

(57) ABSTRACT

Aqueous solutions of the calcium and/or magnesium salts of 3-hydroxy-3-methylbutyric acid are useful for parenteral administration of the calcium and/or magnesium in the treatment and prevention of disorders caused by or accompanied by hypocalcemia or hypomagnesia.

### REFERENCE TO RELATED APPLICATION

This application claims priority to U.S. provisional application Ser. No. 60/411,229, filed on Sep. 17, 2002.

### FIELD OF THE INVENTION

The present invention relates to compositions and methods for preventing and/or treating conditions associated with calcium and/or magnesium deficiencies in humans or other animals. In particular, the invention relates to compositions comprising calcium and/or magnesium salts of 3-hydroxy-3-methylbutyric acid in a form suitable for parenteral administration to a subject in need thereof.

### BACKGROUND OF THE INVENTION

In human and animal metabolism, calcium is essential for the maintenance of the functional integrity of the nervous, muscular, and skeletal systems, as well as cell membrane and capillary permeability. The calcium cation is an important activator in many enzymatic reactions and is essential to a number of physiologic processes including the transmission of nerve impulses; contraction of cardiac, smooth, and skeletal muscles; renal function; respiration; and blood coagulation. Calcium also plays a regulatory role in the release and storage of neurotransmitters and hormones, in the uptake and binding of amino acids, in cyanocobalamin (vitamin B12) absorption and in gastrin secretion.

The calcium of bone is in a constant exchange with the calcium of plasma. Since the metabolic functions of calcium are essential for life, when there is a disturbance in the calcium balance because of dietary deficiency or other causes, the stores of calcium in bone may be depleted to fill the body's more acute needs. Therefore, on a chronic basis, normal mineralization of bone depends on adequate amounts of total body calcium.

Dietary calcium is absorbed from the small intestine. About one third of ingested calcium is absorbed although this can vary depending upon dietary factors and the state of the small intestine.

Following absorption, calcium first enters the extracellular fluid and is then rapidly incorporated into skeletal tissue. Bone contains 99% of the body's calcium; the remaining 1% is distributed equally between the intracellular and extracellular fluids.

Normal total serum calcium concentration ranges from 9-10.4 mg/dL (4.5-5.2 mEq/L), but only ionized calcium is physiologically active. Serum calcium concentrations are not necessarily accurate indications of total body calcium; calcium deficiency (hypocalcemia) can occur even though total body calcium is increased. Of the total serum calcium concentration, 50% is in the ionic form and 5% is complexed by phosphates, citrates, and other anions. Approximately 45% of the serum calcium is bound to plasma proteins; for a change in serum albumin of 1 g/dL, the serum calcium concentration may change about 0.8 mg/dL (0.04 mEq/dL). Concentrations of calcium in the cerebrospinal fluid are about 50% of serum calcium concentrations and tend to reflect ionized serum calcium concentrations.

Symptoms of hypocalcemia include neuromuscular irritability, paraesthiae of the distal extremities and circumoral area, jitteriness, muscle cramps and twitching, laryngospasm, tetany, and seizures. Cardiac manifestations may progress to ventricular fibrillation or heart block.

Common causes of hypocalcemia include hypoparathyroidism, chronic renal insufficiency, acute pancreatitis, septic shock, Vitamin D deficiency, and hypomagnesaemia. Trauma, especially with rapid crush injuries to major muscle groups causing rhabdomyolosis, releases cellular phosphorus, which complexes with calcium and lowers serum calcium levels. Malignant diseases, such as prostate and breast cancers, increase osteoblastic activity, leading to increased bone formation and hypocalcemia. Rapid cell destruction in response to chemotherapy (“tumor-lysis syndrome”) increases serum phosphorus, leading to complexation with serum calcium and hypocalcemia.

Hypocalcemia is common in premature and asphyxiated infants and in the infants of diabetic mothers.

Due to the inefficiency with which orally administered calcium is absorbed, parenteral administration of calcium salts is often required in acute hypocalcaemia and hypocalcaemic tetany.

Intravenous injections of calcium have been used in the treatment of acute renal, biliary, and intestinal colic, and as an adjunct to the treatment of severe hyperkalaemia (excessive blood serum potassium) and as an aid in the treatment of depression due to overdosage of magnesium sulphate. (Calcium is an antagonist of magnesium toxicity).

Calcium increases the force of myocardial contractions and has been used as an inotrope in cardiac resuscitation.

Parenteral administration of calcium is widely employed to prevent hypocalcemia in exchange transfusions where citrated blood is used and in long-term electrolyte replacement therapy.

In veterinary medicine, parenteral administration of calcium is used to treat acute and chronic hypocalcemic conditions in cows, sheep, and goats, especially lactation tetany (“milk fever”).

Parenteral calcium is often administered to treat the bites of venomous spiders and the sting of the puss caterpillar (Megalopyge opercularis).

Acute hypocalcemic emergencies are caused by the ingestion of fluoride compounds and dermal exposure to hydrofluoric acid. Exposure of as little as 2.5% of the surface area of the body can rapidly produce potentially lethal hypocalcemia.

Solutions of calcium salts are irritating to tissue and are seldom injected intramuscularly or subcutaneously. Calcium salts are irritating to tissue when administered by intramuscular or subcutaneous injection and cause mild to severe local reactions including burning, necrosis and sloughing of tissue, cellulitis, soft tissue calcification, and abscess formation; venous irritation may occur with intravenous administration. When injected intravenously, calcium salts must be administered slowly through a small needle into a large vein to avoid extravasation of the calcium-containing solution into the surrounding tissue, with resultant necrosis.

Calcium chloride, CaCl₂, has an elemental calcium content of 36% by weight on a dry basis and is freely soluble in water. However, it is by far the most irritating of all the calcium salts employed for the parenteral administration of calcium; because it is appreciably toxic to veins, it is not suitable for prolonged infusion. For these reasons calcium chloride has been largely supplanted by calcium gluconate and calcium gluceptate.

Calcium gluconate (synonyms: calcium glubionate; D-gluconic acid calcium salt) Ca(C₆H₁₁O₇)₂, 9.3% by wt. elemental calcium on a dry basis, slowly dissolves in 30 parts of water at 20° C., forming a saturated solution with a pH of 6-7. Solutions of calcium gluconate are often supersaturated and stabilized by the addition of calcium saccharate tetrahydrate (elemental calcium content 6.3% by weight).

The solubility of calcium gluconate in water can be increased to 20% by weight or more by adding a complexing agent like boric acid. “Calcium borogluconate” is the designation widely employed for acidic solutions containing mixtures of calcium gluconate and boric acid. In the British Pharmacopoeia and European Pharmacopoeia the ratio of boric acid calcium gluconate is 15.0% to 18.0% by weight, corresponding to an elemental calcium content of 7.3% to 7.9% on a dry basis. Calcium borogluconate is widely used in veterinary medicine for the administration of calcium by injection.

Solutions of calcium gluceptate (synonyms: calcium glucoheptonate; D-glycero-D-glucoheptonoic acid calcium salt), CaC₁₄H₂₆O₁₆, with an elemental calcium content of 8.2% by weight on a dry basis, is also widely employed for the parenteral administration of calcium.

Magnesium is important for the proper functioning of nerves and muscles. An essential constituent of many enzyme systems, particularly those involved with energy generation, it is the fourth most plentiful cation in the body. Magnesium is required by all enzymatic processes involving ATP and is also required by many of the enzymes involved in nucleic acid synthesis.

Like calcium salts, magnesium salts are not well absorbed from the gastro-intestinal tract when administered orally.

Since magnesium is secreted in large amounts in the gastro-intestinal fluid, excessive losses in diarrhea, stoma or fistula are the most common causes of magnesium deficiency (hypomagnesaemia). Magnesium deficiency may also occur in cases of diabetes, chronic alcoholism, or diuretic therapy. It has been reported after prolonged treatment with aminoglycosides.

Hypomagnesaemia is often accompanied by hypocalcemia, with which it may be confused.

Symptoms of magnesium deficiency include anorexia, nausea, vomiting, lethargy, weakness, personality change, cardiac arrhythmia, ventricular fibrillation, cardiac insufficiency, and sudden cardiac death.

Symptomatic hypomagnesaemia is indicated by plasma magnesium levels of less than 0.7 millimoles per liter. Magnesium is given initially by intravenous infusion or by intramuscular injection, typically with an aqueous solution of magnesium sulfate (MgSO₄) or magnesium chloride (MgCl₂). For maintenance (e.g., in intravenous nutrition), parenteral doses of magnesium are of the order of 10 to 20 millimoles of Mg²⁺ daily.

Intravenous administration of magnesium is also recommended for the emergency treatment of serious cardiac arrhythmias, especially in the presence of hypokalaemia (when hypomagnesaemia may also be present) and when salvos of rapid ventricular tachycardia show the characteristic twisting wave front known as torsades de pointes. The usual dose of magnesium sulfate is an intravenous injection of 8 millimoles of Mg²⁺ over 10-15 minutes (repeated once if necessary). Some workers hold the view that magnesium is beneficial if administered immediately in cases of suspected acute myocardial infarction.

Parenteral administration of magnesium has been shown to have a major role in eclampsia for the prevention of recurrent seizures. Regimens vary between hospitals, but typically involve initial intravenous administration of about 16 millimoles of Mg²⁺ over 5-10 minutes followed by intravenous infusion at a rate of 4 millimoles of Mg²⁺ every hour for at least 24 hours after the last seizure.

Parenteral administration of magnesium is employed in the treatment of pregnancy-induced hypertension and pre-term labor.

Monitoring of blood pressure, respiratory rate, and urinary output is carried out, as is monitoring for clinical signs of overdosage (loss of patellar reflexes, weakness, nausea, sensation of warmth, flushing, drowsiness, double vision, and slurred speech). Injections of calcium salts are employed for the management of magnesium toxicity.

### SUMMARY OF THE INVENTION

The present invention provides improved methods for the parenteral administration of calcium and/or magnesium to an animal or human subject in need thereof by administering aqueous solutions comprising calcium and/or magnesium salts of 3-hydroxy-3-methylbutyric acid. Methods of treating hypocalcemia and/or hypomagnesia by administering a therapeutically effective amount of the calcium and/or magnesium salts of 3-hydroxy-3-methylbutyric acid, respectively are also described. The methods of the present invention can also be used to prevent and treat disorders caused by or accompanied by hypocalcemia or hypomagnesia.

More particularly, one aspect of the invention relates to a method for preventing and/or treating a condition associated with calcium and/or magnesium deficiency in a human or other animal subject. The method includes parenterally administering a safe and effective amount of calcium 3-hydroxy-3-methylbutyrate, magnesium 3-hydroxy-3-methylbutyrate or mixtures thereof to a subject. Examples of conditions treated or prevented include, but are not limited to, hypocalcemia and hypomagnesia.

In accordance with another aspect of the present invention, a method for elevating blood serum levels of calcium and/or magnesium cations in a human or other animal subject in need thereof is described. The method includes parenterally administering a safe and effective amount of a composition including at least one of calcium 3-hydroxy-3-methylbutyrate or magnesium 3-hydroxy-3-methylbutyrate to the subject in need of such treatment.

In accordance with another aspect of the present invention, a parenteral composition for elevating blood serum levels of alkaline earth metal cations in a human or other animal subject in need thereof is provided. The composition in certain embodiments includes an alkaline earth metal component, a biocompatible organic acid component, and a sterile aqueous solution. The sterile aqueous solution may be, for example, a normal saline solution, lactated ringers, or dextrose in water. Examples of alkaline earth metals useful in the present invention include, but are not limited to, calcium, magnesium, and mixtures thereof. The biocompatible organic acid is 3-hydroxy-3-methylbutyric acid.

### DESCRIPTION OF THE INVENTION

All documents cited are, in relevant part, incorporated herein by reference; the citation of any document is not to be construed as an admission that it is prior art with respect to the present invention.

As used herein, the term “comprising” means that the described composition or process includes the components or steps recited but is open to the inclusion of additional components or steps. The terms “consisting essentially of” and “consisting of” are embodied in the term “comprising.”

As used herein, the term “pharmaceutically acceptable” refers to a composition that is suitable for use with humans and/or animals without undue adverse side effects (such as toxicity, irritation, or allergic response) commensurate with a reasonable benefit/risk ratio. The term “safe and effective amount” as used herein refers to a quantity of a component which is sufficient to yield a desired therapeutic response without undue adverse side effects commensurate with a reasonable benefit/risk ration when used in the manner of this invention. The specific “safe and effective amount” will vary with such factors as the particular condition being treated, the physical condition of the patient, the duration of the treatment, the nature of any concurrent therapy, and the specific formulations employed.

The term “elemental” as used herein means of or pertaining to the element referred to. The elements involved in the present invention are primarily calcium, magnesium and other minerals beneficial for parenteral administration. Elemental percentage indicates the percentage of elemental calcium, magnesium, etc. present in a composition. Accordingly, the elemental percentage of calcium in calcium carbonate does not include the percentage of carbonate present.

The compositions of the present invention are soluble at high concentrations. The term “soluble” as used herein means capable of being dissolved, going into a liquid state from a solid state. The solubility of a mineral is an indicator of how bioavailable that mineral is. (See Schaafsma, G., “Bioavailability of Calcium and Magnesium,” European J. of Clinical Nutrition, 1977, “It is clear that availability for absorption requires calcium to be solubilized, either in free ionic or complexed form.”) Solubility also plays an important role in the preparation of parenteral compositions.

The calcium salt of 3-hydroxy-3-methylbutyric acid (synonyms: beta-hydroxy-beta-methylbutyric acid; 3-hydroxy-isovaleric acid) forms a monohydrate with the chemical formula Ca(C₅H₉O₃)₂—H₂O. With an elemental calcium content of 13.7% by weight on a dry basis, calcium 3-hydroxy-3-methylbutyrate (“CaHMB”) dissolves readily in water, forming a 27% by weight saturated aqueous solution at 20° C. with a neutral or slightly alkaline pH. In aqueous solutions, calcium 3-hydroxy-3-methylbutyrate dissociates into calcium cations (Ca²⁺) and 3-hydroxy-3-methylbutyrate anions. The 3-hydroxy-3-methylbutyrate anion is a naturally occurring mammalian metabolite; consequently the calcium salt and many other metallic salts of 3-hydroxy-3-methylbutyric acid are non-toxic.

Due to its relatively high content of elemental calcium, CaHMB offers the ability to deliver larger amounts of elemental calcium (as the calcium cation) per unit weight than the calcium salts that are currently widely employed for parenteral administration. This is beneficial because concentrated solutions of salts contain more dissolved solutes than the intercellular and intracellular fluids of the body. Such solutions are “hypertonic,” causing a net flow of water out of cells across their semi-permeable membranes by osmosis. These undesirable effects can be minimized by delivering calcium in solutions with a comparatively high calcium content relative to total solutes.

Calcium gluconate and calcium gluceptate are generally administered parenterally in the form of 10% by weight aqueous solutions, delivering 2.32 millimoles of elemental calcium (in the Ca²⁺ form) per 10 ml and 2.04 millimoles of elemental calcium per 10 ml, respectively. By comparison, 10 ml of a 10% by weight solution of calcium 3-hydroxy-3-methylbutyrate delivers 3.42 millimoles of elemental calcium.

The calcium salt of 3-hydroxy-3-methylbutyric acid can be prepared by reacting a slight (i.e. 5%) stoichiometric excess calcium oxide (“unslaked lime”) or calcium hydroxide (“slaked lime”) with an aqueous solution containing free 3-hydroxy-3-methylbutyric acid, preferably at an elevated temperature of 40 to 70 degrees C. Alternatively, calcium carbonate can be employed. The unreacted excess oxide, hydroxide, or carbonate can be removed by filtration and the calcium salt 3-hydroxy-3-methylbutyric acid is recovered by crystallizing, spray-drying, or freeze-drying (lyophilizing) the resulting filtrate.

The magnesium salt of 3-hydroxy-3-methylbutyric acid (synonyms: beta-hydroxy-beta-methylbutyric acid; 3-hydroxy-isovaleric acid) has the chemical formula Mg(C₅H₉O₃)₂. With an elemental magnesium content of 9.4% by weight on a dry basis, magnesium hydroxymethylbutyrate monohydrate (“MgHMB”) dissolves readily in water, forming a 32% by weight saturated aqueous solution at 20° C. This high degree of solubility corresponds to a high degree of bioavailabilty as well as chemical compatibility when co-administered with the calcium salt of 3-hydroxy-3-methylbutyric acid.

The magnesium salt of 3-hydroxy-3-methylbutyric acid can be prepared by reacting a 5% stoichiometric excess of basic magnesium carbonate (approximate composition (MgCO₃)₄.Mg(OH)₂.5H₂O, equivalent to 40 to 42% MgO) with an aqueous solution containing free 3-hydroxy-3-methylbutyric acid, preferably at an elevated temperature of 40 to 70 degrees C. Alternatively, magnesium oxide or magnesium hydroxide can be employed. The unreacted excess carbonate, oxide, or hydroxide can be removed by filtration and the magnesium salt of 3-hydroxy-3-methylbutyric acid can be recovered by crystallizing, spray-drying, or freeze-drying (lyophilizing) the resulting filtrate.

Both the calcium and the magnesium salts of 3-hydroxy-3-methylbutyric acid are stable powders with long shelf lives. If pure, they dissolve in distilled water to form aqueous solutions of nearly neutral or slightly alkaline pH. They remain highly soluble over a broad range of pH values.

If it is desired to simultaneously co-administer the calcium and magnesium salts of hydroxy-2-methylbutyric acid (e.g., in cases of hypocalcemia and hypomagnesia), they can be synthesized and isolated separately and subsequently blended to produce a composition with the desired ratio of calcium to magnesium. Alternatively, the precursor calcium and magnesium compounds (i.e. calcium and magnesium carbonates or calcium and magnesium oxides or hydroxides) can be mixed in the desired ratio in a preliminary step and co-dissolved in an aqueous solution of 3-hydroxy-3-methylbutyric acid to form a mixture of the calcium and magnesium hydroxy-3-methylbutyrate salts of this invention.

For parenteral administration in the treatment or prevention of chronic or acute hypocalcemia, a solution of calcium 3-hydroxy-3-methylbutyrate in sterile, distilled water can be prepared. The concentration of calcium 3-hydroxy-3-methylbutyrate can range from about 1% to about 27% by weight, but is preferably about 10% by weight. A 10% solution will contain about 13.7 milligrams of elemental calcium (as Ca²⁺) per milliliter.

For the purpose of elevating blood serum levels of calcium cation in accordance with one aspect of the invention, a particularly useful dosage is from about 7 to about 14 millimoles of available calcium cation (corresponding to about 2 to 4 grams of calcium 3-hydroxy-3-methylbutyrate or about 20 to 40 grams of a 10% by weight solution of calcium 3-hydroxy-3-methylbutyrate) for adults, delivered at a rate not exceeding 4.0 milligrams of calcium cation per minute (1 milliliter or less of a 10% by weight solution of calcium 3-hydroxy-3-methylbutyrate every 3.5 minutes). For maintenance by prolonged delivery in intravenous fluid by continuous infusion, a 10% solution of calcium 3-hydroxy-3-methylbutyrate can be diluted and delivered at a rate of about 0.4 to about 3.0 milligrams of elemental calcium (as Ca²⁺) per kilogram of patient body weight per hour. In cases of acute hypocalcemic tetany, an initial intravenous injection of about 10 milliliters of a 10% solution of calcium 3-hydroxy-3-methylbutyrate should be administered, followed by a daily infusion corresponding to 40 milliliters, but plasma calcium should be monitored.

For parenteral administration in the treatment or prevention of chronic or acute hypomagnesia, a solution of magnesium 3-hydroxy-3-methylbutyrate in sterile, distilled water is prepared. The concentration of magnesium 3-hydroxy-3-methylbutyrate can range from about 1% to about 32% by weight, but is preferably about 10% by weight. A 10% solution will contain 9.4 milligrams of elemental magnesium (as Mg²⁺) per milliliter. For the replacement of magnesium in documented cases of magnesium deficiency the preferred dosage is from about 10 to about 20 milliliters of a 10% solution (about 94 to about 188 mg elemental magnesium) per hour for 24 hours, delivered at a rate not exceeding 20 milliliters per hour. For intravenous infusion, a solution of about 8 grams in 250 milliliters of 5% dextrose solution is efficacious. For maintenance in prolonged intravenous fluid, from about 20 to about 40 milliliters of the 10% solution (about 188 to about 376 mg elemental magnesium) can be added to the total intravenous fluid delivered per day; this will deliver 0.3 to 0.7 millimoles of magnesium per hour.

For the intravenous co-administration of calcium 3-hydroxy-3-methylbutyrate and magnesium 3-hydroxy-3-methylbutyrate, a solution containing both calcium 3-hydroxy-3-methylbutyrate and magnesium 3-hydroxy-3-methylbutyrate in sterile, distilled water is prepared. The total concentration of the combined solutes can range from about 1% to about 20% by weight, but is preferably about 10% by weight. The ratio of calcium 3-hydroxy-3-methylbutyrate to magnesium 3-hydroxy-3-methylbutyrate can range from about 100:1 to about 1:100 by weight, but is preferably about 20:1 to about 2:1 by weight for most clinical applications. For the prevention of hypocalcemia and hypomagnesia in exchange transfusions and in long-term electrolyte replacement therapy, these solutions can be further diluted or added to the total daily intravenous fluid supplement.

The compositions of the present invention may be diluted as is known to those skilled in the art. In accordance with certain embodiments, a physiologically acceptable diluent may be formed of physiologic saline solution containing a carbohydrate such as, for example, glucose (or dextrose) or sorbitol. Specific examples of physiologically acceptable diluents include WFI (water for injection), D5W (5% dextrose in water), and D5W 0.45% saline which can be used to form a pharmaceutical formulation suitable for patient administration.

The following non-limiting examples illustrate the compositions, processes and uses of the present invention.

### EXAMPLE 1

74.1 grams (1.0 moles) of calcium hydroxide is added to approximately 400 ml of water with vigorous agitation, forming a slurry. 224 grams of 3-methyl-3-hydroxy-3-methylbutyric acid (1.90 moles) is introduced slowly. Following the addition of the acid, the mixture is heated to 70 degrees C. and stirred for 90 minutes, then allowed to cool to room temperature. Insoluble particles of excess lime are removed by filtration on a Buechner funnel through Whatman #4 (20 micron) filter paper. The filtrate, a clear, non-turbid liquid, is evaporated to dryness, producing crystalline calcium 3-hydroxy-3-methylbutyrate monohydrate in virtually quantitative yield.

In a separate vessel, 20.15 grams (0.50 moles) of magnesium oxide is added to approximately 200 ml of water with vigorous agitation, forming a slurry. 112 grams of 3-methyl-3-hydroxy-3-methylbutyric acid (0.95 moles) is introduced slowly. Following the addition of the acid, the mixture is heated to 70 degrees C. and stirred for three hours, then allowed to cool to room temperature. Insoluble particles of excess magnesium oxide are removed by filtration on a Buechner funnel through Whatman #4 (20 micron) filter paper. The filtrate, a clear, non-turbid liquid, is evaporated to dryness, producing crystalline magnesium 3-hydroxy-3-methylbutyrate in virtually quantitative yield.

The calcium and magnesium salts are ground together in a mortar and pestle to produce a mixture with a bulk density of 0.60 grams per cubic centimeter.

### EXAMPLE 2

74.1 grams (1.0 moles) of calcium hydroxide and 20.15 grams (0.50 moles) of magnesium oxide are added to 500 ml of water with vigorous agitation, forming a slurry. 336 grams of 3-methyl-3-hydroxy-3-methylbutyric acid (2.85 moles) is introduced slowly. Following the addition of the acid, the mixture is heated to 70 degrees C. and stirred for 90 minutes, then allowed to cool to room temperature. Insoluble particles of excess lime and magnesia are removed by filtration on a Buechner funnel through Whatman #4 (20 micron) filter paper. The filtrate, a clear, non-turbid liquid, is evaporated to dryness, producing an intimate mixture of crystalline calcium 3-hydroxy-3-methylbutyrate monohydrate and magnesium 3-hydroxy-3-methylbutyrate in virtually quantitative yield.

What is claimed is:

1. A method for elevating blood serum levels of calcium and/or magnesium cations in a human or other animal subject in need thereof comprising:
parenterally administering a safe and effective amount of a composition comprising at least one salt selected from the group consisting of calcium 3-hydroxy-3-methylbutyrate and magnesium 3-hydroxy-3-methylbutyrate to said subject.

2. The method of claim 1 wherein said composition comprises calcium 3-hydroxy-3-methylbutyrate.

3. The method of claim 2 wherein said composition contains calcium 3-hydroxy-3-methylbutyrate at a concentration of from about 1% to about 27% by weight.

4. The method of claim 3 wherein said composition is administered at a dosage providing from about 7 to about 14 millimoles of available calcium cation.

5. The method of claim 4 wherein said dosage is administered at a rate not exceeding 40 mg of calcium cation per minute.

6. The method of claim 2 wherein said step of administering a safe and effective amount of the composition comprises:
administering said composition at a rate to deliver from about 0.4 to about 3.0 mgs of elemental calcium per kg of subject weight per hour.

7. The method of claim 1 wherein said composition comprises magnesium 3-hydroxy-3-methylbutyrate.

8. The method of claim 7 wherein said composition contains magnesium 3-hydroxy-3-methylbutyrate at a concentration of from about 1% to about 32% by weight.

9. The method of claim 8 wherein said composition is administered to a subject suffering from magnesium deficiency at a dosage providing from about 94 to about 188 mg of elemental magnesium per hour for a period of time sufficient to increase serum levels of magnesium to normal levels.

10. The method of claim 8 wherein said step of administering a safe and effective amount of the composition comprises:
administering said composition at a rate to deliver from about 188 to about 376 mg of elemental magnesium per day.

11. The method of claim 10 wherein said composition delivers a sufficient amount of magnesium to a subject in need thereof to maintain magnesium levels at acceptable levels during prolonged intravenous fluid delivery.

12. The method of claim 1 wherein said composition comprises calcium 3-hydroxy-3-methylbutyrate and magnesium 3-hydroxy-3-methylbutyrate.

13. The method of claim 12 wherein said composition comprises from about 100:1 to about 1:100 calcium 3-hydroxy-3-methylbutyrate to magnesium 3-hydroxy-3-methylbutyrate by weight.

14. The method of claim 13 wherein said composition comprises from about 20:1 to about 2:1 calcium 3-hydroxy-3-methylbutyrate to magnesium 3-hydroxy-3-methylbutyrate by weight.

15. A method for preventing and/or treating a condition associated with calcium and/or magnesium deficiency in a human or other animal subject comprising parenterally administering to said subject a safe and effective amount of a composition comprising at least one salt selected from the group consisting of calcium 3-hydroxy-3-methylbutyrate and magnesium 3-hydroxy-3-methylbutyrate.

16. The method of claim 15 wherein said condition is selected from the group consisting of hypocalcemia, hypomagnesia, hyperkalemia, overdosage of magnesium sulfate, lactation tetany, exchange transfusions, long-term electrolyte replacement therapy, cardiac resuscitation, bites of venomous spiders, stings of puss caterpillars, cardiac arrhythmia, eclampsia, recurrent seizures, pregnancy-induced hypertension, pre-term labor and combinations thereof.

17. The method of claim 15 wherein said composition comprises calcium 3-hydroxy-3-methylbutyrate.

18. The method of claim 17 wherein said composition contains calcium 3-hydroxy-3-methylbutyrate at a concentration of from about 1% to about 27% by weight.

19. The method of claim 18 wherein said composition is administered at a dosage providing from about 7 to about 14 millimoles of available calcium cation.

20. The method of claim 17 wherein said step of parenterally administering a safe and effective amount of the composition comprises:
administering said composition at a rate to deliver from about 0.4 to about 3.0 mgs of elemental calcium per kg of subject weight per hour.

21. The method of claim 15 wherein said composition comprises magnesium 3-hydroxy-3-methylbutyrate.

22. The method of claim 21 wherein said composition contains magnesium 3-hydroxy-3-methylbutyrate at a concentration of from about 1% to about 32% by weight.

23. The method of claim 22 wherein said composition is administered to a subject suffering from hypomagnesia at a dosage providing from about 94 to about 188 mg of elemental magnesium per hour for a period of time sufficient to increase serum levels of magnesium to normal levels.

24. The method of claim 22 wherein said step of administering a safe and effective amount of the composition comprises:
administering said composition at a rate to deliver from about 188 to about 376 mg of elemental magnesium per day.

25. The method of claim 24 wherein said composition delivers a sufficient amount of magnesium to a subject in need thereof to maintain magnesium levels at acceptable levels during prolonged intravenous fluid delivery.

26. The method of claim 15 wherein said composition comprises calcium 3-hydroxy-3-methylbutyrate and magnesium 3-hydroxy-3-methylbutyrate.

27. The method of claim 26 wherein said composition comprises from about 100:1 to about 1:100 calcium 3-hydroxy-3-methylbutyrate to magnesium 3-hydroxy-3-methylbutyrate by weight.

28. The method of claim 27 wherein said composition comprises from about 20:1 to about 2:1 calcium 3-hydroxy-3-methylbutyrate to magnesium 3-hydroxy-3-methylbutyrate by weight.

29. A composition for elevating blood serum levels of alkaline earth metal cations in a human or other animal subject in need thereof comprising:
(a) an alkaline earth metal component,
(b) a biocompatible organic acid component, and
(c) a sterile aqueous solution selected from the group consisting of normal saline, lactated ringers, and 5% dextrose in water,
wherein said alkaline earth metal is selected from the group consisting of calcium, magnesium, and mixtures thereof, and said biocompatible organic acid comprises 3-hydroxy-3-methylbutyric acid,
wherein said composition is suitable for parenteral administration to said subject.

30. The composition of claim 29 wherein said sterile aqueous solution is normal saline.

31. The composition of claim 29 wherein said alkaline earth metal is calcium.

32. The composition of claim 31 wherein the alkaline earth metal is present at a concentration of from about 1.4 mg per milliliter to about 37 milligrams per milliliter.

33. The composition of claim 31 wherein the alkaline earth metal is present at a concentration of about 13.7 milligrams per milliliter.

34. The composition of claim 29 wherein said alkaline earth metal is magnesium.

35. The composition of claim 34 wherein the alkaline earth metal is present at a concentration of from about 0.9 mg per milliliter to about 30 milligrams per milliliter.

36. The composition of claim 34 wherein the alkaline earth metal is present at a concentration of about 9.4 milligrams per milliliter.

37. The composition of claim 29 wherein the alkaline earth metal component is a mixture of calcium and magnesium.

38. The composition of claim 37 wherein the ratio of calcium to magnesium is from about 1:100 to about 100:1 by weight.

39. The composition of claim 37 wherein the ratio of calcium to magnesium is from about 20:1 to about 2:1 by weight.
